(19) United States
(12) Patent Application Publication
(10) Pub. No.: US 2012/0073580 A1
(43) Pub. Date: Mar. 29, 2012

(54) STIMULATING CONDOM

(75) Inventors: BENG SIM CHUAH, PETALING JAYA (MY); BURTON VAN ROOYEN, VICTORIA (AU); JAMIE ASHWORTH, FREEHOLD, NJ (US); KC NGUYEN, DOTHAN, AL (US); CHAYAPORN PONGTHANOMSAK, A. PHUNPHIN SURATTHANI (TH); NGO CHOON YONG, SELANGOR DARUL EHSAN (MY); BUNJONG KENGKHETKORN, SURATTHANI (TH)

(73) Assignee: ANSELL LIMITED, Victoria (AU)

(21) Appl. No.: 13/243,038

(22) Filed: Sep. 23, 2011

Related U.S. Application Data
(60) Provisional application No. 61/385,694, filed on Sep. 23, 2010.

(51) Int. Cl. A61F 6/04; B28B 1/14

(52) U.S. Cl. 128/844; 264/299

(57) ABSTRACT

Condoms are disclosed, which include an open end defining a base, a closed end defining an apex, a tubular sheath extending from the base to the apex, a tip portion, adapted to cover a glans of a penis, disposed adjacent to the apex, a bottom portion extending from the base portion to the tip portion, the tubular sheath including an inside surface defining a circumference; and a plurality of integrally formed ribs or protrusions disposed on at least one of the tip portion, the base portion, or the bottom portion of the tubular sheath and forming an undulating pattern around the circumference.

### CROSS REFERENCE TO RELATED APPLICATIONS

This application claims priority from U.S. Provisional patent application Ser. No. 61/385,694, filed Sep. 23, 2010, the disclosure of which is hereby incorporated by reference in its entirety.

### BACKGROUND

1. Field of the Invention

Embodiments of the present invention generally relate to condoms and, more particularly, to condoms having shapes, features, and surface textures imparting enhanced stimulation during use and methods of making such condoms.

2. Description of the Related Art

Condoms and other prophylactics and protective devices provide physical barriers against the transmission of bodily and other fluids. Condoms are typically made of an elastomeric layer. Typical condom designs include a shaft portion formed by a tubular sheath with an open end and a closed end. Examples of areas of appreciably heightened nerve sensitivity are found on the male body at the frenulum of prepuce of the penis, which is positioned on the underside of the penis. The frenulum is described as very reactive and particularly responsive to touch including touch that is described as light and soft. Repeated stimulation of the frenulum is thought to cause orgasm and ejaculation in most men. Examples of areas of appreciably heightened nerve sensitivity are found on the female body at the vagina-labium minor interface, and the clitoris.

Designers have attempted to create condoms having textures and patterns to provide stimulation. For example, condoms have been studded or ribbed. And, some condoms have been textured by heat- and pressure-embossing patterns onto films, and then forming the condom. However, a condom of this structure requires several additional steps and does not contain grooves of significant depth or protrusions of significant height for stimulation.

Consequently, a condom that provides protection against the transmission of bodily fluids and other fluids, while imparting enhanced stimulation during use to both males and females, would present a significant advance in the art.

### SUMMARY

Condoms are disclosed, which include an open end defining a base, a closed end defining an apex, a tubular sheath extending from the base to the apex, a tip portion, adapted to cover a glans of a penis, disposed adjacent to the apex, a bottom portion extending from the base portion to the tip portion, the tubular sheath including an inside surface defining a circumference, and a plurality of integrally formed ribs or protrusions disposed on at least one of the tip portion, the base portion, or the bottom portion of the tubular sheath and forming an undulating pattern around the circumference.

### BRIEF DESCRIPTION OF THE DRAWINGS

So that the manner in which the above recited features of the present invention can be understood in detail, a more particular description of embodiments of the invention, briefly summarized above, may be had by reference to embodiments, some of which are depicted in the appended drawings. It is to be noted, however, that the appended drawings illustrate only examples of embodiments of the invention and are therefore not to be considered limiting of its scope because the invention admits to other equally effective embodiments.

FIG. 1 depicts a perspective view of a condom, according to one or more embodiments of the present invention;

FIG. 2 depicts a side elevational view of the condom depicted in FIG. 1;

FIG. 3 depicts an enlarged partial view of the condom shown in FIG. 2;

FIG. 4 depicts a cross-sectional view of the condom depicted in FIG. 2, taken along line 4-4, including a base portion;

FIG. 5 depicts an enlarged partial cut-away view of a base portion of the condom shown in FIG. 4;

FIG. 6 depicts a front view of the condom shown in FIG. 2;

FIG. 7 depicts a rear view of the condom shown in FIG. 2; and

FIG. 8 depicts a top view of a former according to one or more embodiments of the present invention.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of the present invention pertain to a condom having a tubular sheath and a plurality of ribs or protrusions disposed thereon. In some embodiments, the plurality of ribs or protrusions may form an undulating pattern around the circumference and at least a portion of the length of the tubular sheath. The plurality of ribs or protrusions may be disposed on the tubular sheath and have a structure adapted to stimulate the areas of heightened nerve stimulation found on both the male and female body, for example, the frenulum, the vagina-labium minor interface, and the clitoris. The structure and disposition of the plurality of ribs or protrusions is unexpected in condoms because they are formed on condoms using a typical dipping process, wherein a former comprising the desired texture is dipped into a bath of a latex composition. Other dipping technologies may be utilized, such as solvent dipping. The elastomeric properties of the latex composition and known formers inhibit the formation of such ribs which provide such stimulation, without the use of molding processes or processes other than dipping processes.

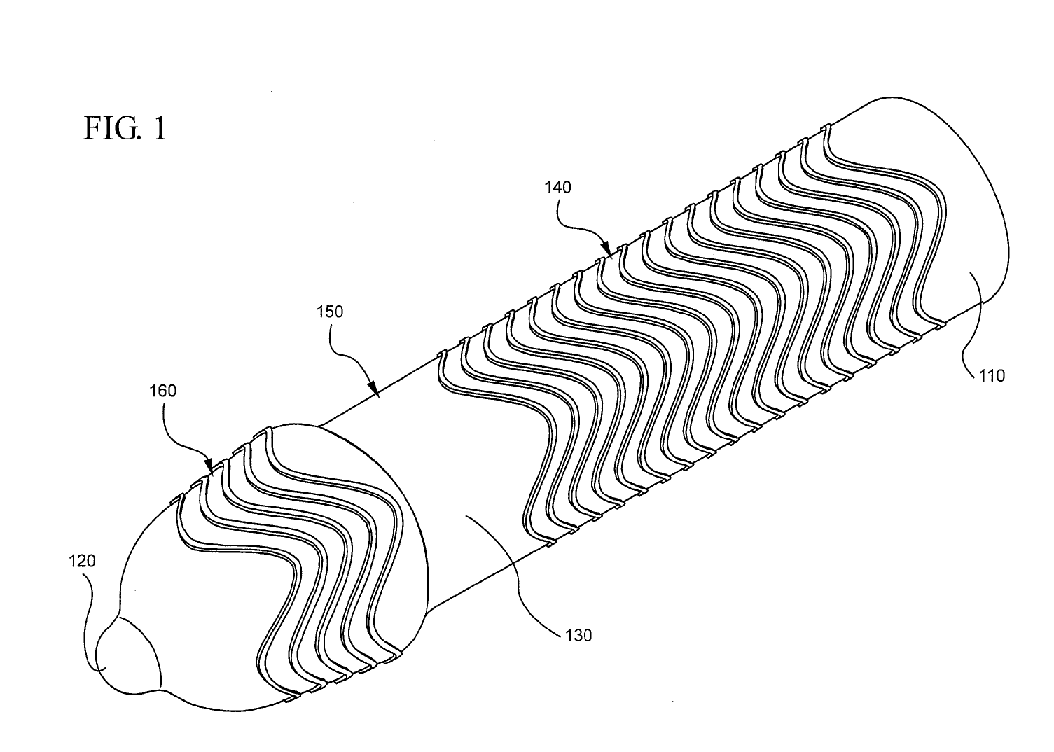

Embodiments of condoms according to the invention are shown in FIGS. 1-7. FIG. 1 depicts a perspective view of a condom, according to one or more embodiments of the present invention. Condom 100 includes an open end 110, a closed end 120, and a tubular sheath 130 extending therebetween. The tubular sheath 130 includes an axial length and a surface defining a circumference. In one or more embodiments, tubular sheath 130 has a thickness in the range from about 0.030 mm to about 0.150 mm, excluding the height of the plurality of ribs or protrusions (e.g., studs, mounds, dots, and the like) 170 disposed thereon. Ribs can be defined as elongate members forming a raised rail, which can further comprise an undulating pattern around the circumference of the condom. Protrusions can be defined as raised, rounded, convex mounds, dots, or studs, the pattern of which may also comprise an undulating pattern around the circumference of the condom. Dots can be defined as round, rectangular, square, or diamond. In one or more embodiments, tubular sheath 130 has a thickness in the range from about 0.040 mm to about 0.120 mm, excluding the height of the plurality of ribs or protrusions 170 integrally formed thereon. In one or more embodiments, the thickness of the tubular sheath increases from open end 110 to closed end 120. As will be discussed below, the plurality of ribs or protrusions 170 disposed on tubular sheath 130 may increase the overall thickness of tubular sheath 130, where the thickness or height of the plurality of ribs or protrusions 170 is included. For example, the plurality of ribs or protrusions 170 may have a thickness or height of about 0.4 mm to 1.0 mm; and more specifically 0.5 mm. In such embodiments, tubular sheath 130 may have a thickness of about 0.540 mm to about 0.620 mm at locations at which the plurality of ribs or protrusions 170 is disposed.

In one or more embodiments, tubular sheath 130 is formed from an elastomeric material that may include an elastomeric rubber. The elastomeric rubber may be a synthetic rubber, natural rubber latex, and combinations, mixtures, or blends thereof. Other examples of suitable elastomeric materials include synthetic polyisoprene, guayule, polyurethane, polyethylene, copolymers, block copolymers, and blends, mixtures, or combinations thereof. The term “natural rubber latex” as used in this disclosure encompasses cured elastomeric material sourced from Hevea brasiliensis (the traditional rubber tree), Parthenium argentatum (guayule), sunflower, goldenrod, and the like, as well as genetically modified variations of these or other biological sources. The term “integrally formed” means a condom in which the tip portion, middle portion, and base portion, including all ribs, protrusions, mounds and other features, are formed into a single unit from one manufacturing process.

As shown in FIG. 1, tubular sheath 130 includes a base portion 140 that is disposed adjacent to open end 110 and extends from the open end 110 toward closed end 120. Tubular sheath 130 also includes a middle portion 150 extending from base portion 140 toward a tip portion 160 that is disposed adjacent to closed end 120. The combination of the base portion and the middle portion can be termed the “bottom portion.”

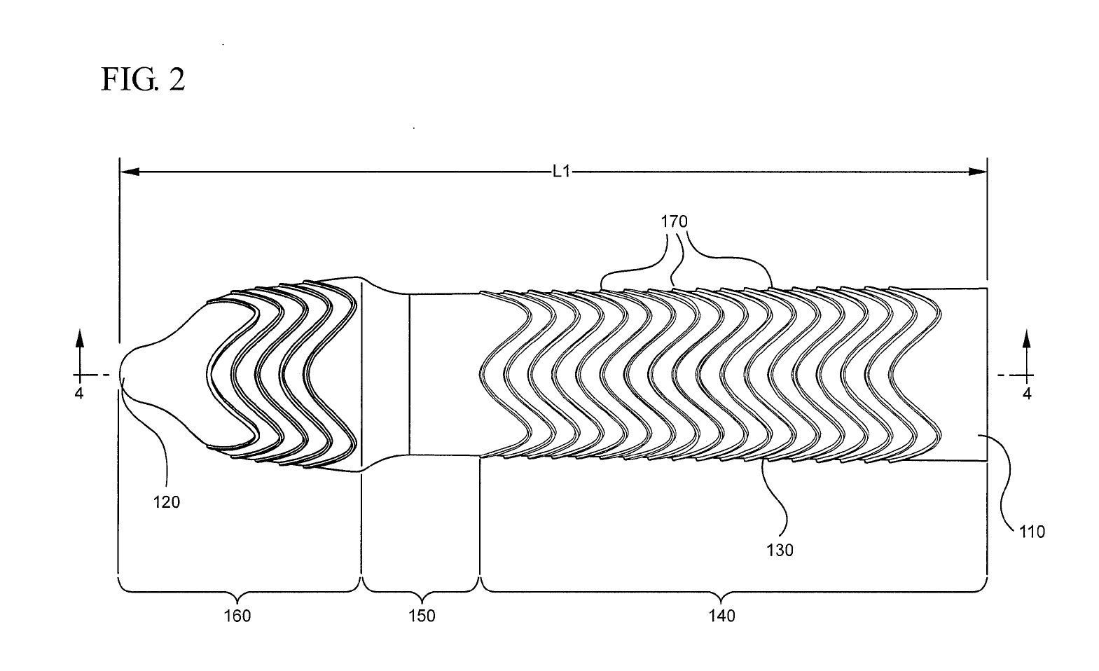

FIG. 2 depicts a side elevational view of the condom depicted in FIG. 1. Tubular sheath 130 in embodiments of the invention have a length L1 from open end 110 to closed end 120 in the range from about 150 mm to about 230 mm. In one or more embodiments, the length of tubular sheath 130 may be approximately 215 mm.

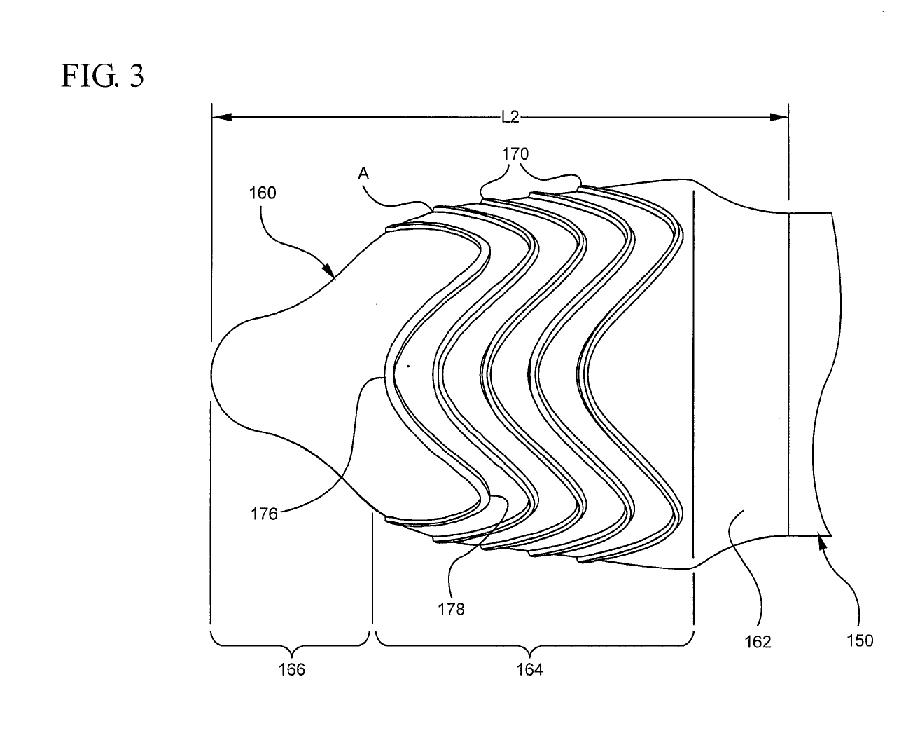

FIG. 3 depicts an enlarged partial view of the condom shown in FIG. 2, and shows, more clearly, tip portion 160 having a length L2, for example, in the range from about 50 mm to about 70 mm. In another embodiment, tip portion 160, adapted to fit over the glans, may have a length L2 in the range from about 55 mm to about 65 mm. In some embodiments, the length of tip portion 160 may be approximately 60 mm. Tip portion 160 of one embodiment includes a first region 162 disposed adjacent to middle portion 150 of the tubular sheath and a third region 166 disposed adjacent to closed end 120 of the tubular sheath. Where there are ribs or protrusions on both base portion 140 and tip portion 160, in certain embodiments there is middle portion 150 that is free of ribs or protrusions. Tip portion 160 also includes a second region 164 disposed between first region 162 and third region 166.

In certain embodiments, the first region 162 may also be described as a narrowed region or narrowed section and includes a cross-sectional width, i.e., diameter, increasing from the middle portion 150 to second region 164. The cross-sectional width of first region 162 may be in the range from about 33 mm to approximately 43 mm. In another embodiment, the cross-sectional width of first region 162 is approximately 40 mm. In other embodiments, the cross-sectional width of first region 162 at its base may be approximately 39 mm, 39.25 mm, 39.5 mm, 39.75 mm, 40 mm, 40.25 mm, 40.5 mm, 40.75 mm, 41 mm, 41.25 mm, 41.5 mm, 41.75 mm, 42 mm, 42.25 mm, 42.5 mm, or 42.75 mm. The “base” of a region is the end closest to open end 110. In another embodiment, the cross-sectional width of first region 162 may be in the range from about 40 mm to about 43 mm. The cross-sectional width of first region 162 may increase in a non-linear manner from middle portion 150 to second region 164. In one embodiment, the cross-sectional width of first region 162 increases at a greater rate at a portion of first region 162 adjacent to second region 164 relative to a portion of first region 162 adjacent to middle portion 150. The length of first region 162 is in the range from about 10 mm to about 15 mm. In one or more embodiments, the combined length of first region 162 and second region 164 is in the range from about 40 mm to about 50 mm. In some embodiments, the combined length of first region 162 and second region 164 is approximately 46.5 mm.

In some embodiments, the cross-sectional width of tip portion 160 may be at a maximum at second region 164, which may be described as an enlarged region or enlarged section. In one embodiment, the cross-sectional width of tip portion 160 is at a maximum at the base of second region 164. The cross-sectional width of second region 164 decreases from its base toward closed end 120. In one or more embodiments, the cross-sectional width of second region 164 at its base may be in the range from about 35 mm to about 45 mm. The cross-sectional width of second region 164 of one or more embodiments may be 41 mm or less. In one or more embodiments, the cross-sectional width of second region 164 may be approximately 41 mm, when measured at the base of second region 164.

In one or more embodiments, the cross-sectional width of second region 164 decreases substantially linearly from its base to the third region 166 boundary (its “apex”). In some embodiments, the cross-sectional width of second region 164 decreases substantially linearly along a portion of second region 164. In at least one embodiment, the cross-sectional width of second region 164 decreases substantially linearly from approximately 41 mm at its base to a point A disposed near third region 166.

The third region 166 may be described as a tapered region or tapered section. The third region 166 has a cross-sectional width that decreases from its base to closed end 120. In certain embodiments, the cross-sectional width of the third region 166 decreases at a greater rate than the cross-sectional width of second region 164. The cross-sectional width of the third region of one or more embodiments may decrease in a non-linear manner. In one or more embodiments, the rate of decrease of the cross-sectional width of third region 166 may be greater at a portion of third region 166 adjacent its base than at a portion of third region 166 adjacent to its apex. In one or more alternative embodiments, the rate of decrease of the cross-sectional width of third region 166 may be greater at a portion of third region 166 adjacent to its apex than at a portion adjacent to its base. The cross-sectional width of third region 166 may have a cross-sectional width at its base in the range from about 18 mm to about 35 mm. In one or more embodiments, third region 166 may have a cross-sectional width of about 21 mm.

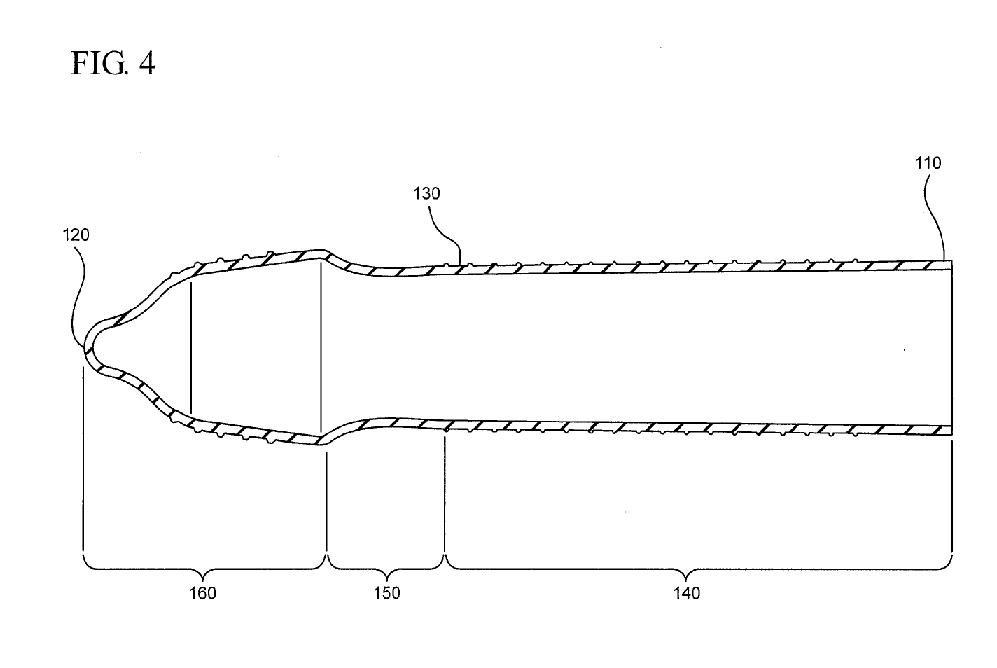

FIG. 4 depicts a cross-sectional view of the condom depicted in FIG. 2, taken along line 4-4 including a base portion, showing base portion 140 and middle portion 150, which have, for example, a combined length in the range from about 95 mm to about 135 mm. Alternatively, base portion 140 and middle portion 150 may have a combined length of about 120 mm. In one or more embodiments, base portion 140 may have a length that is greater than the length of middle portion 150. The base portion 140 of one or more embodiments may have a length in the range from about 90 mm to about 120 mm. In some embodiments, the length of base portion 140 may be about 105 mm. The length of middle portion 150 may be adjusted. In one or more embodiments, tubular sheath 130 may not have middle portion 150 and include only base portion 140 and tip portion 160. In certain embodiments, middle portion 150 has no protrusions or ribs, and has its base at a point at the rib or protrusion of base portion 140 furthest from open end 110. In certain embodiments, the apex of middle portion 150 is the circumference point adapted to match, roughly, the retroglandular sulcus of a user's penis.

The open end 110 of the condom has a cross-sectional width of, for example, about 33 mm or more. In one or more embodiments, the cross-sectional width of open end 110 may be approximately 33 mm. In some embodiments, open end 110 of the condom has a cross-sectional width in the range from about 33 mm to about 38 mm. The upper limit for the cross-sectional width of open end 110 of one or more embodiments may be approximately 40 mm. In one or more embodiments, open end 110 of the condom may be larger, however, it will be understood that the remaining portions of the condom may also be larger in a proportional manner as understood in the art. The cross-sectional width of tubular sheath 130 decreases along base portion 140 and middle portion 150 from open end 110 to tip portion 160. In one or more embodiments, the cross-sectional width of tubular sheath 130 decreases substantially linearly from open end 110 to tip portion 160.

In one or more embodiments, tubular sheath 130 includes a plurality of ribs or protrusions 170. Specifically, the plurality of ribs or protrusions 170 are formed on a surface of tubular sheath 130 and form an undulating pattern around the circumference of tubular sheath 130. In one or more embodiments, the plurality of ribs or protrusions 170 is disposed along the entire axial length of tubular sheath 130 or along one or more portions of the axial length of tubular sheath 130. In one or more embodiments, the plurality of ribs or protrusions 170 is disposed at regular intervals along tubular sheath 130, including the tip, base, and middle portions. In one or more embodiments, the plurality of ribs or protrusions 170 is disposed at irregular intervals along tubular sheath 130, including the tip, base, and middle portions. In some embodiments, the plurality of ribs or protrusions 170 are discrete ribs or protrusions completely encircling the circumference of tubular sheath 130.

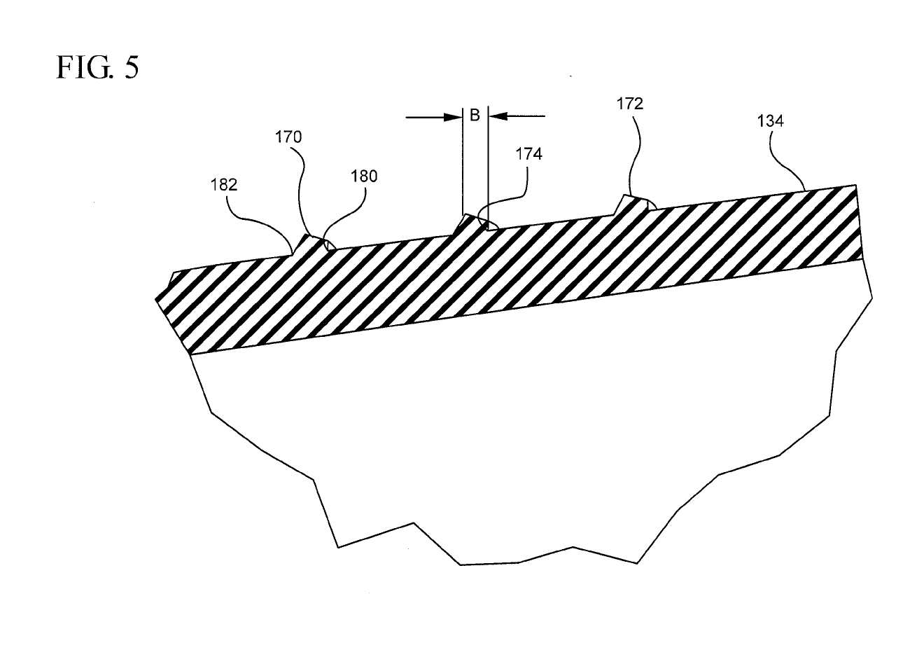

FIG. 5 depicts an enlarged partial cut-away view of base portion of the condom shown in FIG. 4, showing tubular sheath 130, which includes an outside surface 134 and an inside surface 132 (as more clearly shown in FIGS. 5 and 7). The plurality of ribs or protrusions 170 of one or more embodiments is disposed on the outside surface 134. The inside surface 132 of such embodiments may be free of any ribs or may have a flat or uniform surface. In one or more alternative embodiments, the plurality of ribs or protrusions 170 may be disposed on inside surface 132, while the outside surface is free of ribs. Optionally, the plurality of ribs or protrusions 170 may be disposed on outside surface 134 and inside surface 132 or alternatively, the plurality of ribs or protrusions 170 may extend through tubular sheath 130 so they are disposed on both inside surface 132 (not shown) and outside surface 134. In some embodiments, the plurality of ribs or protrusions 170 are disposed on outside surface 134 of tubular sheath 130 and extend inwardly toward inside surface 132 of tubular sheath 130.

In any of the embodiments in accordance with the invention, the plurality of ribs or protrusions 170 may be disposed along at least a portion of axial length of tubular sheath 130 at tip portion 160 and at base portion 140. In one or more alternative embodiments, the plurality of ribs or protrusions 170 may be disposed only along all or a portion of base portion 140, middle portion 150 or tip portion 160. In some embodiments, the plurality of ribs or protrusions 170 may be disposed on base portion 140 and middle portion 150, while tip portion 160 is free of ribs or protrusions. Optionally, the plurality of ribs or protrusions 170 may be disposed on middle portion 150 while base portion 140 and tip portion 160 are free of ribs or protrusions or the plurality of ribs or protrusions may be disposed on middle portion 150 and tip portion 160 while base portion 140 is free of ribs. In this case, the middle portion is measured from the apex of one rib or protrusion furthest away from the base of the rib or protrusion in the undulating pattern.

In one or more embodiments, as shown in FIG. 5, each of the plurality or ribs or protrusions 170 may have a uniform thickness B when measured along the axial length of tubular sheath 130. A rib 170 can be defined as a contiguous structure of raised elevation relative to outside surface 134. A protrusion can be defined as a raised dot or circular mound relative to outside surface 134. In one or more alternative embodiments, the plurality of ribs or protrusions 170 has a different thickness, which can vary depending on the position of the rib or protrusion. In one or more embodiments, a plurality of ribs or protrusions 170 disposed on tip portion 160 may have a thickness that is greater than or less than a plurality of ribs disposed on middle portion 140 or base portion 150. Alternatively, the thickness of the plurality of ribs or protrusions 170 may vary from one rib or protrusion to the next rib or protrusion. For example, the thickness of the ribs or protrusion may alternate such that a first rib has a first thickness, a second rib disposed adjacent to the first rib may have a second thickness that is greater than the first thickness, and a third rib disposed on the opposite side of the second rib from the first rib and may have the first thickness. As shown in FIG. 5, the plurality of ribs 170 has a thickness B in the range from about 0.35 mm to about 2 mm. In another embodiment, the thickness of the plurality of ribs is in the range from about 0.50 mm to about 1 mm.

Each of the plurality of ribs or protrusions 170 may have an outer end 174 disposed at outside surface 134 of tubular sheath 130 and an inner end 172 disposed at the opposite end of the rib from outer end 174. In one or more embodiments, the thickness of the plurality of ribs 170 varies from outer end 174 to inner end 172. In the embodiment shown, the thickness of the plurality of ribs 170 shown in FIG. 5 at outer end 174 is greater than the thickness of the plurality of ribs 170 at inner end 172. In one or more alternative embodiments, the thickness of the plurality of ribs or protrusions 170 at outer end 174 may be less than the thickness of the plurality of ribs 170 at inner end 172. Optionally, the variations in the thickness of the plurality of ribs from outer end 174 to inner end 172 may vary from rib to rib or between different groups of ribs disposed at different portions of the axial length of tubular sheath 130.

Each of the plurality of ribs or protrusions 170 have a height or cross-sectional width measured from outer end 174 to inner end 172 in the range from about 0.03 mm to about 3 mm. In one or more embodiments, the height or cross-sectional width of the plurality of ribs or protrusions 170 may be in the range from about 0.05 mm to about 2.5 mm. In one or more specific embodiments, each of the plurality of ribs or protrusions 170 may have a cross-sectional width or height measured from the outside surface of the tubular sheath of approximately 0.1 mm, 0.2 mm, 0.3 mm, 0.4 mm, 0.5 mm, 0.6 mm, 0.7 mm, 0.8 mm, 0.9 mm, and 1 mm. In certain embodiments, the upper limit of the cross-sectional width or height of the plurality of ribs or protrusions 170 may include 2 mm, 2.2 mm, 2.4 mm, 2.6 mm and 2.8 mm. The height or cross-sectional width of the plurality of ribs or protrusions 170 may vary from along the thickness of the rib. In some embodiments, each of the plurality of ribs or protrusions 170 includes a first end 180 and a second end 182, wherein the thickness of the rib generally increases from first end 180 to second end 182. In one or more embodiments, the thickness of the ribs may decrease from first end 180 to second end 182. The height or cross-sectional width of the plurality of ribs or protrusions 170, measured from outer end 174 to inner end 172, may vary from rib to rib or between different groups of ribs disposed at different portions of the axial length of tubular sheath 130.

The thickness and the height or cross-sectional width of the plurality of ribs or protrusions 170 are related due to the nature of the dipping process by which the condom is formed and may be modified in this regard. For example, increases in the thickness of the plurality of ribs or protrusions 170 may be accompanied by a decrease in the height or cross-sectional width of the plurality of ribs or protrusions 170. In addition, the properties of the elastomeric material utilized to form tubular sheath 130 may also influence the thickness and/or height of the plurality of ribs or protrusions 170. For example, the height of the plurality of ribs or protrusions 170 may be adjusted so that air bubbles are not formed in the elastomeric material during the dipping process. Moreover, the thickness and/or height of the plurality of ribs or protrusions 170 may also be adjusted to control the amount of elastomeric material disposed between the ribs.

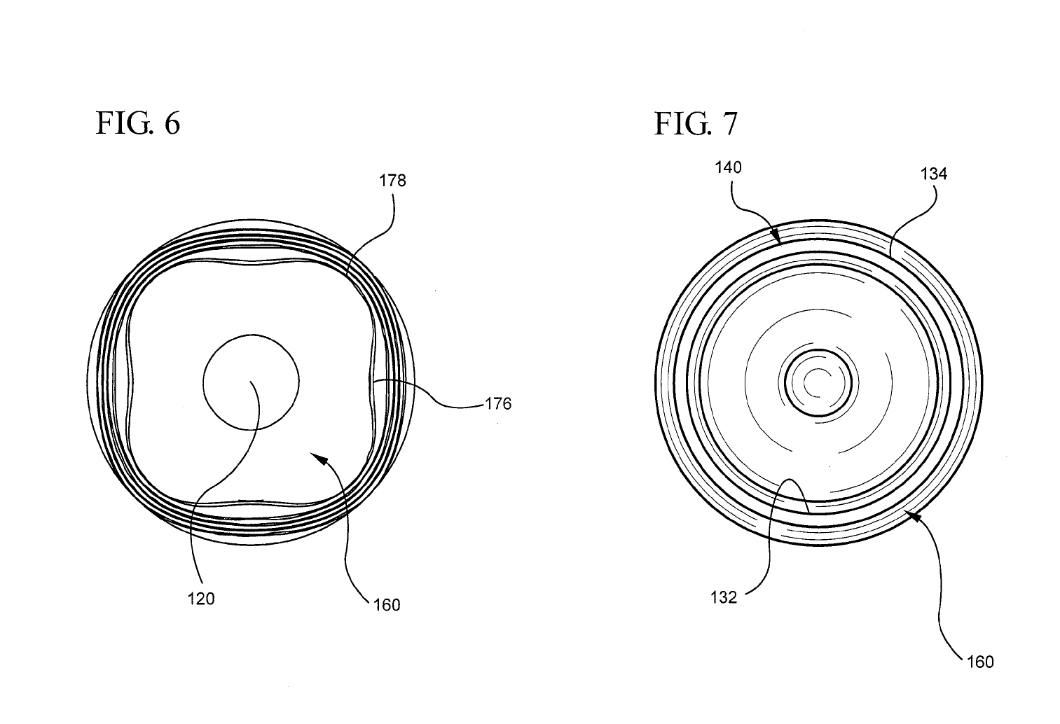

In some embodiments, each of the plurality of ribs or protrusions 170 forms an undulating pattern that includes or provides at least one crest 176 and at least one trough 178. The plurality of ribs or protrusions 170 shown in FIGS. 1-7 have a plurality of crests 176 and troughs 178. FIG. 6 depicts a front view of the condom shown in FIG. 2. FIG. 7 depicts a rear view of the condom shown in FIG. 2. Each of FIGS. 6-7 show a plurality of crests and troughs that alternate along the circumference of tubular sheath 130, forming a wave-like pattern. In other words, if a longitudinal circular axis were drawn through the middle of a given pattern, the plurality of ribs or protrusions would extend further away from the axis, reach a maximum distance from the axis, change direction, return to the axis, and while continuing in the same direction, become further away from the axis, subsequently change direction again and return to the axis. The plurality of crests 176 and troughs 178 of each rib are shown in FIGS. 1-7 as aligned with each other. In one or more alternative embodiments, the plurality of crests 176 and troughs 178 of each rib may be disposed at different locations along the circumference of the tubular sheath from one another. The distance between the plurality of crests 176 of a single rib may be constant or irregular and may be equal to or different than the distance between the plurality of troughs 178. Where protrusions are not continuous, for example, mounds or raised dots, the mounds may still define patterns such as outlined in the preceding paragraph. The pattern is present if readily visually apparent. If the undulations of different patterns replicate, the pattern may be apparent with less density of protrusions. Alternately, if the patterns are less duplicative, great “pixel” density may be required for the patterns to be apparent.

In one or more embodiments, the distance between the plurality of crests 176 and the plurality of troughs 178 on a single rib is constant. In some embodiments, the distance between the plurality of crests 176 and the plurality of troughs 178 is in the range from about 3 mm to about 20 mm. In one or more embodiments, the distance between the plurality of crests 176 and the plurality of troughs 178 is in the range from about 10 mm to about 16 mm. In one or more embodiments, the distance between the plurality of crests and the plurality of troughs of a single rib increases as the cross-sectional width of the tubular sheath increases. In one or more alternative embodiments, the distance between the plurality of crests 176 and the plurality of troughs 178 may be adjusted to provide different stimulation. For example, the distance can be increased at one area of the tubular sheath to provide different stimulation. The remaining ribs disposed at different areas of the tubular sheath may have a different distance between the plurality of crests and plurality of troughs.

In one or more embodiments, the plurality of ribs 170 is not equally spaced from each other. In one or more embodiments, the distance between the ribs disposed next to each other varies depending on the position of the ribs along the axial length of tubular sheath 130. For example, in embodiments in which the plurality of ribs 170 are disposed on tip portion 160, the distance between the ribs may decrease along the length of the tubular sheath toward closed end 120. In embodiments in which the plurality of ribs 170 are disposed along base portion 140, the distance between the plurality of ribs 170 may decrease toward open end 110 or, in other words, may increase toward closed end 120. In one or more embodiments, the distance between the ribs is about 2 mm to 20 mm; and more specifically 10 mm.

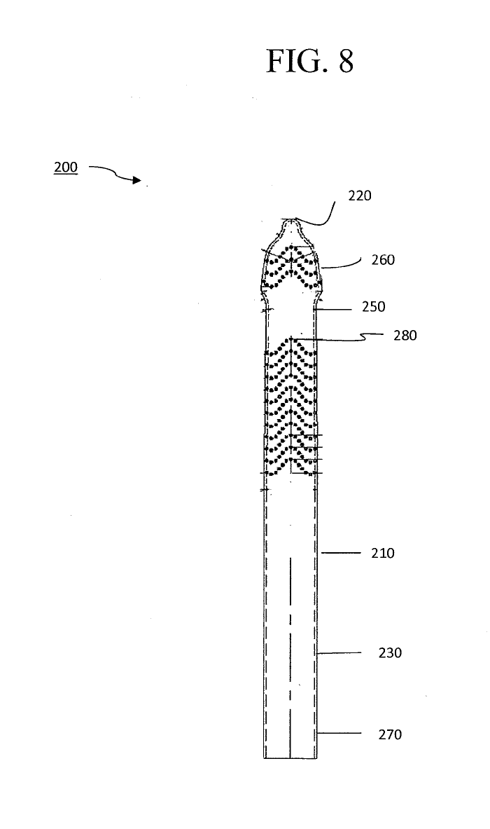

Methods for forming condoms are also included in accordance with embodiments of the present invention. In one or more embodiments, at least one method includes providing a former comprising an axial length, a circumference, and a plurality of depressions, ribs, or protrusions disposed along at least a portion of the length and around or along the circumference of the former. The method of one or more embodiments includes disposing an elastomeric layer on the former, generally by a dipping process, to form a tubular sheath, as otherwise described herein, and curing the elastomeric layer to form a cured elastomeric layer. Referring to FIG. 8, former 200 may include an outside surface 210 and a plurality of concave dimples or depressions 280 disposed on outside surface 210 and extending inwardly from the outside surface 210 of former 200. The former 200 may include a first end 230, a second end 220, a base segment 270 disposed adjacent to the first end 230, a tip segment 260 disposed adjacent to the second end 220, and a middle portion 250 extending between base segment 270 and tip segment 260. In additional, the distance of the concave dimples or depressions within a line/curve/wave from one to the other is about 2.0 mm to about 5.0 mm; and more specifically 4.0 mm.

In one or more embodiments, the step of disposing an elastomeric layer on the former includes coating the former with a coagulant component and dipping the coated former into a bath or tank containing an elastomeric composition as otherwise described herein. In one or more embodiments, the former may be dipped in a bath or tank containing an elastomeric composition without the use of a coagulant component. Other methods of disposing an elastomeric layer on the former may be utilized, such as solvent dipping. The temperature of the elastomeric composition may be controlled, as is known in the art, and may include additives to control or modify the properties of the elastomeric composition. The elastomeric composition of one or more embodiments may also include a cure package or vulcanization agents to promote cross-linking during the curing process. As the former is dipped into the bath or tank, the dwell time and immersion and extraction speeds of the dipping process may be controlled and modified to adjust thickness of the resulting elastomeric layer that forms the tubular sheath. In one or more embodiments, the elastomeric material disposed on the former is cured or otherwise treated to form a cured elastomeric layer. In one or more embodiments, the elastomeric layer is dried in ambient air and heated to a temperature in the range from about 50° C. to about 150° C. The resulting elastomeric layer or tubular sheath replicates the shape of the former. In one or more embodiments, the method utilizes a former that includes a plurality of depressions disposed on the surface thereof to form the plurality of ribs or protrusions on embodiments of the condom described above. The former may be formed from a ceramic material, metallic material, or other material known in the art.

In one or more embodiments, the former may include a tubular body having a first end and a second end. The tubular body may have an overall shape that is similar to the shape of one or more embodiments of the tubular sheath of the condom described above. The tubular body of the former may include a base segment that is disposed adjacent to the first end and extends from the first end toward the second end. In one or more embodiments, the second end is utilized to form closed end 120 of the condom described above, while the first end of the former is utilized to form base portion 140 of the condom described above. The tubular body of the former according one or more embodiments may also include a middle segment that extends from the base segment toward a tip segment that is disposed adjacent to the second end.

The tubular body of the former may have a length that is greater than about 150 mm. In one or more embodiments, the length of the tubular body is greater than about 180 mm or alternatively, greater than about 200 mm. In one or more embodiments, the length of the tubular body may be in the range from about 300 mm to about 450 mm. In one or more specific embodiments, the length of the tubular body may be about 360 mm.

In one or more embodiments, the tip segment may have a length in the range from about 55 mm to about 65 mm. In one or more embodiments, the length of the tip segment may be approximately 60 mm. The tip segment of the former may include a contoured shape that is similar to the shape of tip portion 160 described above with reference to the former. Specifically, the tip segment may include a first segment that is disposed adjacent to the middle segment of the tubular body and a third segment disposed adjacent to the second end of the tubular body. The tip segment of the former may also includes a second segment disposed between the first segment and the third segment.

The first segment of the tubular body of the former may also be described as a narrowed region or narrowed section and includes a cross-sectional width that increases from the middle segment of the tubular body to the second segment of the tip segment 260. The cross-sectional width of the first segment of one or more embodiments may be in the range from about 33 mm to approximately 43 mm. In another embodiment of the former, the cross-sectional width of first region 162 is approximately 40 mm or more. In one or more specific embodiments, the cross-sectional width of the first region 162 may be 39 mm, 39.25 mm, 39.5 mm, 39.75 mm, 40 mm, 40.25 mm, 40.5 mm, 40.75 mm, 41 mm, 41.25 mm, 41.5 mm, 41.75 mm, 42 mm, 42.25 mm, 42.5 mm, or 42.75 mm. In some embodiments, the cross-sectional width of first region 162 may be in the range from approximately 40 mm to approximately 43 mm. The cross-sectional width of the first segment may increase in a non-linear manner from the middle segment to the second segment. In one embodiment, the cross-sectional width of the first segment may increase and decrease as described above with respect to the cross-sectional width of first region 162 of the condom. The length of the first segment is in the range from about 10 mm to about 15 mm. In one or more embodiments, the combined length of the first segment and the second segment is in the range from about 40 mm to about 50 mm. In one or more other embodiments, the combined length of the first segment and the second segment of the former is approximately 46.5 mm.

The cross-sectional width of the tip segment may be the greatest at the second segment, which may be described as an enlarged region or enlarged section. Specifically, the cross-sectional width of the tip segment is the greatest at the point at which the first segment and the second segment meet. The cross-sectional width of the second segment of one or more embodiments decreases from the point at which the second segment meets the first segment toward the second end, as described with reference to second region 166 of the condom described above. The cross-sectional width of the second segment of one or more embodiments may be in the range from about 35 mm to about 45 mm. In one or more embodiments, the cross-sectional width of the second segment may be 41 mm or less. In one or more embodiments, the cross-sectional width of the second segment at the widest portion of the tip portion may be approximately 41 mm measured adjacent to the first segment.

The third segment may be described as a tapered region or tapered section. The third segment has a cross-sectional width that decreases from second segment to the second end of the former. The cross-sectional width of the third segment decreases as described with reference to the third region 166 of the condom described above. In one or more embodiments, the cross-sectional width of the third segment may be in the range form about 18 mm to about 35 mm. In one or more other embodiments, the cross-sectional width of the third segment may be about 21 mm or less.

The base segment and the middle segment of the former may have a combined length that permits formation of a condom having a base portion 140 and middle portion in the range from about 235 mm to about 395 mm. In one or more embodiments, the length of the base segment and the middle segment may be in the range from about 295 mm to about 305 mm. The length of the middle segment of the former may be adjusted. In one or more embodiment, the tubular body of the former may be free of a middle segment and include only the base segment and the tip segment.

The first end of the former utilized to form the open end 110 of the condom may have a cross-sectional width of about 33 mm or more. In one or more embodiments, the cross-sectional width of the first end may be approximately about 33 mm. In some embodiments, the first end of the former has a cross-sectional width in the range from about 33 mm to about 38 mm. The upper limit for the cross-sectional width of the first end of one or more embodiments may include 40 mm. In one or more embodiments, the first end of the former may be larger, however, it will be understood that the remaining portions of the former may also be larger in a proportional manner as understood in the art. The cross-sectional width of the tubular body may decreases long the base segment and/or the middle segment from the first end to the tip segment. In one or more embodiments, the cross-sectional width of the tubular body may decrease substantially linearly or constantly from the first end to the tip segment.

In one or more embodiments of the former, the tubular body includes a plurality of concave dimples or depressions that protrude inwardly from an outside surface of the tubular body and form the plurality of ribs or protrusions on the tubular sheath of the condom described herein. The plurality of depressions disposed on the tubular body forms an undulating pattern around the circumference of the tubular body. In one or more embodiments, the plurality of depressions may be disposed along the tubular body or segments of the tubular body to form a plurality of ribs or protrusions 170 on the condom at desired locations along the tubular sheath. In one or more embodiments, the plurality of depressions may be disposed along the entire axial length of the tubular body or along one or more portions of the axial length of the tubular body of the former. In one or more embodiments, the plurality of depressions is disposed at regular intervals along the tubular body of the former. In one or more embodiments, the plurality of depressions is disposed at irregular intervals along the tubular body of the former. Each of the plurality of depressions of one or more embodiments may be discrete depressions that completely encircle the circumference of the tubular body.

In one or more embodiments of the former, the plurality of depressions may be disposed along at least a portion of axial length of the tubular body at the tip segment and at the base segment. In one or more alternative embodiments, the plurality of depressions may be disposed only along all or a portion of the base segment, the middle segment or the tip segment. In another embodiment, the plurality of depressions may be disposed on the base segment and the middle segment, while the tip segment remains free of any depressions such that the resulting condom formed using such a former may have a plurality of ribs or protrusions 170 disposed on base portion 140 and middle portion 150, while tip portion 160 is free of ribs or protrusions. Optionally, the plurality of depressions may be disposed on the middle segment of the former while the base segment and the tip segment are free of depressions such that the resulting condom has a plurality of ribs or protrusions 170 disposed on middle portion 150 and base portion 140 and tip portion 160 are free of any ribs or protrusions. In one or more alternative embodiments, the plurality of protruding ribs may be disposed on the middle segment and the tip segment while the base segment is free of depressions such that the resulting condom has a plurality of ribs disposed on middle portion 150 and tip portion 160, while base portion 140 remains free of any ribs.

In one or more embodiments, each of the plurality of depressions has a uniform thickness when measured along the axial length of the tubular body. In one or more alternative embodiments, the plurality of depressions has different thicknesses, which can vary depending on the position of the depression. In one or more embodiments, a plurality of ribs disposed on the tip segment of the former may have a thickness that is greater than or less than the thickness of a plurality of protrusions disposed on the middle segment or the base segment. Alternatively, the thickness of the plurality of depressions may vary from one depression to the next depression. For example, the thickness of the depressions may alternate to form ribs with a thickness that alternates, as described above. In one or more embodiments, the plurality of depressions may have a thickness in the range from about 0.45 mm to about 2 mm. In some embodiments, the thickness of the plurality of depressions may be in the range from about 0.75 mm to about 1 mm.

The thickness of the plurality of depressions may also vary from one end of each depression to the opposite end of the depression, as described with reference to the plurality of ribs or protrusions 170. In one or more embodiments, the variations in the thickness of the plurality of depressions may differ between different groups of depressions disposed at different portions of the axial length of the tubular body of the former.

Each of the plurality of depressions may have a depth or cross-sectional width measured from an outside surface of the tubular body in the range from about 0.03 mm to about 3 mm. In one or more embodiments, the depth or cross-sectional width of the plurality of depressions is in the range from about 0.025 mm to about 2.5 mm, when measured from the outside surface. In one or more embodiments, each of the plurality of ribs may have a cross-sectional width or height measured from the outside surface of the tubular sheath of approximately 0.4 mm, 0.5 mm, 0.6 mm, 0.7 mm, 0.8 mm, 0.9 mm, or 1 mm. The upper limit of the cross-sectional width or height of the plurality of ribs may include 2 mm, 2.2 mm, 2.4 mm, 2.6 mm, or 2.8 mm. The depth or cross-sectional width of the plurality of depressions may vary from along the thickness of the depression. The depth or cross-sectional width of the plurality of depressions may vary from depression to depression or between different groups of depressions disposed at different portions of the axial length of the tubular body of the former. The thickness and the depth or cross-sectional width of the plurality of depressions may be varied to control the effects of the dipping process on the thickness and depth of the plurality of ribs or protrusions 170.

In some embodiments, each of the plurality of depressions forms an undulating pattern that includes or provides at least one crest and at least one trough, as described with reference to the plurality of ribs of the condom. The plurality of depressions may also have a plurality of crests and troughs that alternate along the circumference of the tubular body to form a wave-like pattern. The plurality of crests and troughs of each depression may be aligned with each other. In one or more alternative embodiments, the plurality of crests and troughs of each rib are disposed at different locations along the circumference of the tubular body from one another. The distance between the plurality of crests of a single depression may be constant and may be equal to distance between the plurality of troughs of the depression.

In one or more embodiments, the distance between the plurality of crests and the plurality of troughs of a single depression is constant. In one or more embodiments, the distance between the plurality of crests and the plurality of troughs is in the range from about 3 mm to about 20 mm. In one or more alternative embodiments, the distance between the plurality of crests and the plurality of troughs is in the range from about 10 mm to about 16 mm.

In one or more embodiments, the plurality of depressions is not equally spaced from each other. In one or more embodiments, the distance between the depressions disposed next to each other varies depending on the position of the depressions along the axial length of the tubular body of the former. For example, in embodiments in which the plurality of ribs or protrusions 170 are disposed on tip portion 160, the distance between the depressions decreases along the length of the tubular sheath toward the second end utilized to form closed end 120 of the condom. In embodiments in which the plurality of depressions are disposed along the base segment, the distance between the plurality of depressions decreases toward the first end utilized to form open end 110 of the condom or, in other words, increases toward the second end utilized to form closed end 120 of the former. In one or more embodiments, the distance between the depressions is about 2 mm or more. In one or more embodiments, the distance between the plurality of depressions is in the range from about 2 mm to about 5 mm. In a more specific embodiment, the distance between the plurality of depressions is about 4 mm. The undulating pattern formed by the plurality of depressions may include at least one crest and at least one trough. In one or more specific embodiments, the undulating pattern includes a plurality of crests and a plurality of troughs, wherein the distance between the plurality of crests and troughs on a single depression is constant and the distance between the plurality of crests on a single depression is constant. In one or more embodiments, the plurality of depressions has a thickness measured along the axial length of the former in the range from about 0.45 mm to about 2 mm. The plurality of depressions of one or more embodiments has a depth or a cross-sectional width measured from the outside surface of the former in the range from about 0.03 mm to about 3 mm. Each depression disposed on the former of one or more embodiments completely encircles the circumference of the former.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the following appended claims and their equivalents.

What is claimed is:

1. A condom comprising:
an open end defining a base;
a closed end defining an apex;
a tubular sheath extending from the base to the apex, comprising:
a tip portion, adapted to cover a glans of a penis, disposed adjacent to the apex;
a bottom portion extending from the base portion to the tip portion, and having an axial length, the tubular sheath including an inside surface defining a circumference; and
a plurality of integrally formed ribs or protrusions disposed on at least one of the tip portion, the base portion, or the bottom portion of the tubular sheath and forming an undulating pattern around the circumference.

2. The condom of claim 1, wherein the tubular sheath comprises an outside surface and the plurality of ribs extend radially outwardly from the outside surface of the tubular sheath.

3. The condom of claim 1, wherein the tubular sheath includes an inside surface and the plurality of ribs extend radially inwardly from the outside surface.

4. The condom of claim 1, wherein the tip portion comprises a cross-sectional shape that includes a first region adjacent to the bottom portion, a second region adjacent to the first region and a third region disposed between the second region and the closed end, wherein the second region has a cross-sectional width that decreases from the base toward the apex.

5. The condom of claim 4, wherein the first region has a cross-sectional width that increases from the base to the apex.

6. The condom of claim 5, wherein the undulating pattern comprises at least one crest and at least one trough.

7. The condom of claim 6, wherein the distance between the crests and the troughs is from about 3 mm to about 20 mm.

8. The condom of claim 6, wherein the at least one rib includes a first rib or protrusion and a second rib or protrusion disposed adjacent to the first rib or protrusion, the distance between the at least one crest of first rib or protrusion and the crest of the second rib or protrusion is at least 2 mm.

9. The condom of claim 8, wherein the distance between the crests of the first rib or protrusion and the ribs or protrusions of the second rib increases from the base to the apex.

10. The condom of claim 1, wherein the each of the plurality of ribs or protrusions has a thickness in the range from about 0.45 mm to about 2 mm and a cross-sectional width measured from the outside surface in the range from about 0.03 mm to about 3 mm.

11. A method for forming a condom comprising:
providing a former comprising an axial length, a circumference, and a plurality of depressions disposed along at least a portion of the axial length, the depressions extending in an undulating pattern around the circumference;
disposing an elastomeric layer on the former to form a tubular sheath; and
curing the elastomeric layer to form a cured elastomeric layer.

12. The method of claim 11, wherein the former comprises an outside surface, a base end, an apex end, a bottom segment disposed adjacent to the base end, and a tip segment disposed adjacent to the apex end, wherein the plurality of depressions extend inwardly from the outside surface.

13. The method of claim 12, wherein the plurality of depressions are disposed on at least one of the tip segment, or the bottom portion of the former, the plurality of depressions forming a undulating pattern around the circumference of the former.

14. The method of claim 13, wherein the tip segment comprises a cross-sectional shape that includes a narrowed section adjacent to the bottom portion, an enlarged section adjacent to the narrowed section and a tapered section disposed between the enlarged section and the apex end, wherein the enlarged section has a cross-sectional width that decreases from the narrowed section toward the second end.

15. The method of claim 14, wherein the narrowed section has a cross-sectional width that increases from the bottom portion to the enlarged section.

16. The method of claim 12, wherein the distance between the plurality of depressions varies and is at least 2 mm.

17. The method of claim 13, wherein the undulating pattern comprises at least two crests and at least two troughs.

18. The method of claim 13, wherein the undulating pattern comprises a plurality of crests and a plurality of troughs, wherein the distance between the plurality of crests and troughs on a single depression is constant and the distance between the plurality of crests on a single depression is constant.

19. The method of claim 13, wherein the distance between the plurality of crests and the plurality of troughs of a single protrusion is in the range from approximately 8 mm to approximately 20 mm.

20. The method of claim 13, wherein the each of the plurality of depressions has a thickness in the range from approximately 0.45 mm to approximately 2 mm and a cross-sectional width measured from the outside surface in the range from approximately 0.03 mm to approximately 3 mm.
